(19) United States
(12) Patent Application Publication
(10) Pub. No.: US 2011/0282243 A1
(43) Pub. Date: Nov. 17, 2011

(54) SWAB

(75) Inventor: Hiroshi NAKATANI, Tondabayashi-shi (JP)

(73) Assignee: SANYO CO., LTD., Osaka (JP)

(21) Appl. No.: 13/100,377

(22) Filed: May 4, 2011

(30) Foreign Application Priority Data
May 10, 2010 (JP) 2010-108534

(51) Int. Cl. A61B 5/00; A61F 11/00; A45D 40/26; A61M 35/00

(52) U.S. Cl. 600/580; 604/1; 606/162; 132/320

(57) ABSTRACT

Provided is a swab 1 in which a fiber lump portion 2 is disposed at one end or both ends of a stick 4, and an adhesive layer is disposed partially or entirely on the surface of the fiber lump portion 2, and a flocked layer 3 covering the adhesive layer is disposed by flocking fiber thereon in a brush shape. The swab 1 has a soft texture and excellent elasticity, and also has superior fluid retention performance and fluid release performance. Therefore, the swab is suitable for taking biological specimens, applying chemical agents to the affected parts, cleaning human bodies or equipment (for example, ear cleaning), applying cosmetic materials, or the like.

### TECHNICAL FIELD

The present invention relates to a flocked swab and, in particular, a swab suitable for taking biological specimens, applying chemical agents to affected parts, cleaning human bodies or equipment (for example, ear cleaning), applying cosmetic materials (for example, mascara), or the like.

### BACKGROUND ART

Conventionally, in the medical field such as diagnosis and clinical analysis, swabs are widely used for taking specimens from the oral cavity, nasal cavity, urethra of patients, or the like. In these swabs, a fiber lump portion is usually formed by winding and adhering fibers, such as cotton, around the tip of a stick, such as a paper stick or a wood stick, in order to make it easier to take fluid specimens.

The conventional swabs of this type have excellent capability of taking a specimen by absorption, and hence have the advantage of taking the necessary amount of the specimen by allowing the specimen to speedily penetrate into the fiber lump portion. On the other hand, however, the fiber lump portion has strong retention force with respect to the penetrated fluid, and hence suffers from the problem of poor specimen release performance. That is, the taken specimen is not sufficiently released when the specimen is transferred to an inspection medium, or the like.

To solve this problem, patent documents 1 and 2 have proposed to use the swabs in which short fibers are flocked at one end of a stick made of plastic or the like by flocking process. These fibers flocked by flocking process are flocked in a brush shape, and are therefore considered to have excellent capability of taking specimens, and superior specimen release performance.

### Prior Art Documents

### Patent Documents

Patent document 1: Japanese Unexamined Patent Publication No. 2007-523663

Patent document 2: Japanese Patent No. 4339906

### SUMMARY OF THE INVENTION

### Problems to be Solved by the Invention

In the swabs described in these two patent documents 1 and 2, the short fibers are adhered to one end of the stick by flocking process. Therefore, the one end of the stick is formed into a round head shape in order to avoid damaging the patient or giving the patient irritation, such as pain, during taking the specimen. However, even when formed into this shape, the stick itself is formed by using a hard material such as plastic, thus resulting in poor elasticity. Therefore, these swabs may give pain or strong irritation to the patient.

Although the swabs having the short fibers adhered to the stick surface by flocking process have the advantage of a soft texture because the fibers are adhered in the brush shape, the swabs cannot be used for cleaning human bodies, such as ear cleaning, or for cleaning irregular surfaces because of the poor elasticity described above.

Accordingly, the main object of the present invention is to provide a swab having a soft texture and excellent elasticity, and also having superior fluid retention performance and fluid release performance.

Other object of the present invention is to provide a swab suitable for uses, such as taking biological specimens, applying chemical agents to the affected parts, cleaning human bodies or equipment, and applying cosmetic materials.

### Means for Solving the Problems

As a result of the extensive research of the inventor of the present invention in order to solve the above problems, the inventor has finally succeeded in providing the swab having excellent elasticity and nice texture by disposing a fiber lump portion at one end of a stick, and by disposing a brush-shaped flocked layer on the surface of the fiber lump portion.

That is, the swab of the present invention includes a stick; a fiber lump portion disposed at one end or both ends of the stick; and a flocked layer covering the surface of the fiber lump portion by flocking thereon fibers, which are the same as or different from those of the fiber lump portion, in a brush shape.

The fibers which form the flocked layer are preferably short fibers composed of one kind or a mixture of two or more kinds selected from the group consisting of rayon, polyester, polyamide, carbon fiber, cotton and silk, or conductive fibers.

An adhesive layer is disposed on the surface of the fiber lump portion, and one end of each of the fibers for forming the flocked layer is adhered to the surface of the fiber lump portion by the adhesive layer.

### Effect of the Invention

According to the present invention, the fiber lump portion having excellent elasticity is disposed at one end or both ends of the stick, and the flocked layer having the fibers flocked in the brush shape is disposed on the surface of the fiber lump portion. This provides the effect of obtaining the swab having elasticity and a velvet textured surface.

Additionally, in the flocked layer, the fibers are flocked substantially vertically in the brush shape. This provides the effect that the flocked layer has excellent fluid retention performance and fluid release performance.

Hence, the swab of the present invention is suitable for uses, such as taking biological specimens, applying chemical agents to the affected parts, cleaning human bodies or equipment, and applying cosmetic materials such as mascara.

### BRIEF DESCRIPTION OF THE DRAWINGS

FIG. 1 is a partially cutaway side view showing the main part of the swab according to one embodiment of the present invention;

FIG. 2 is a schematic explanatory view showing an example of the flocking process in the present invention; and

FIG. 3 is a schematic side view showing the main part of the swab according to other embodiment of the present invention.

### EMBODIMENTS FOR CARRYING OUT THE INVENTION

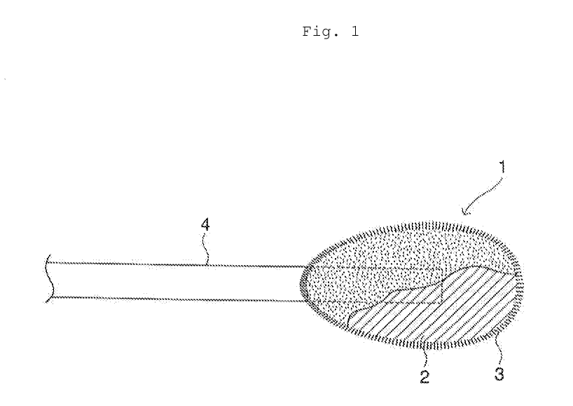

As shown in FIG. 1, in the swab 1 of the present invention, the fiber lump portion 2 is disposed at one end of the stick 4, and the surface of the fiber lump portion 2 is covered with the flocked layer 3.

As the stick 4, it is possible of use a paper stick in which paper having adhesive applied to one surface thereof is wound without forming cavities, as well as a plastic stick, a wood stick, or the like. The stick 1 has a diameter of 1 mm or more, preferably about 1 to 3 mm.

Cotton fibers may be cited as a representative example of the fibers forming the fiber lump portion 2, without being limited thereto, and various kinds of natural fibers or chemical fibers may be used singly or mixed with the cotton fibers. The fiber lump portion 2 is formed by winding and adhering a fiber body, which is an aggregation of the fibers, around the stick, in the same manner as the conventional manufacturing of swabs.

The fiber lump portion 2 can be disposed at one end or both ends of the stick 1. The fiber lump portion 2 usually has an axial length of about 12 to 16 mm, and has a thickness of 2 to 10 mm, preferably about 5 to 7 mm, without being limited thereto. The shape of the fiber lump portion 2 is not limited to the shape as shown in FIG. 1. Depending on the use, the fiber lump portion 2 can be formed into various kinds of optional shapes by subjecting the fiber lump portion 2, together with one end of the stick 4 located therein, to pressure forming, or the like. As the optional shape, it is possible to employ a flat or curved plate shape, such as spatula shape, circular cylinder, circular cone, globular form, drop shape, or a shape formed by stacking a plurality of spherical bodies (namely, a stepped shape with a plurality of circumferential grooves formed therein), or the like.

Examples of the natural fiber include hemp, silk, wool, besides cotton. Examples of the chemical fiber include regenerated fibers such as rayon, cupra, and polynosic; semisynthetic fibers such as acetate and triacetate; and synthetic fibers such as polyamide (nylon), polyester, acryl, polyvinyl chloride, polyurethane, polyvinyl alcohol (vinylon), polyethylene, and polypropylene.

The flocked layer 3 is disposed by allowing short fibers (piles) to be adhered to the surface of the obtained fiber lump portion 2 in the brush shape. As the flocking method, flocking process (electrostatic flocking method) using static electricity can be used. That is, adhesive is previously applied to the surface of the fiber lump portion 2, and the short fibers are adhered in the brush shape (specifically, substantially vertically) to the surface of the fiber lump portion 2 by using electrostatic suction force. Subsequently, the short fibers are fixed by drying or curing the adhesive. Thus, the surface of the fiber lump portion 2 can be covered with the flocked layer 3 of the short fibers.

Examples of the short fiber include rayon, polyester, polyamide, carbon fiber, cotton, and silk. These short fibers can be used singly, or by mixing two or more kinds together. The short fibers can be dyed in the desired color.

The fiber length of the short fibers is preferably about 0.3 to 3 mm, and the fiber thickness thereof is preferably about 0.1 to 5.6 dtex. When the fibers are thin and have a long fiber length, the flocked layer having a good texture can be obtained, and is therefore suitable for uses such as ear cleaning. The kind, fiber length, and thickness of the short fibers may be properly selected depending on the use.

The short fibers are usually subjected to the treatment called electrodeposition treatment. In the electrodeposition treatment, when the short fibers are placed between a pair of electrodes to which a DC high voltage is applied, the short fibers are charged up to a predetermined quantity of electricity so as to become charged. The electrodeposition treatment method can be carried out according to the ordinary method by using the electrodeposition treatment agent conventionally used for flocking process.

Alternatively, in the present invention, as the short fibers, conductive fiber may be used solely, or by mixing with other short fiber. Although no particular limitation is exerted on the conductive fiber, there is, for example, “Belltron” (registered trademark) manufactured by KB SEIREN, LTD. The Belltron is manufactured by performing conjugate melt spinning of two ingredients for a conductive layer in which carbon black or a white metal compound is blended as a conductive fine particle, and for a polymer layer of polyester, polyamide, or the like which protects the conductive layer.

The use of the conductive fiber of this type provides the advantage of eliminating the need for the electrodeposition treatment, and also suppresses the occurrence of static electricity on the surface of the flocked layer 3. In the case of mixing with other short fiber, the conductive fiber is preferably 10 to 90% by weight with respect to the total amount of the mixture.

When the flocked layer 3 is disposed on the surface of the fiber lump portion 2 by flocking process, firstly, an adhesive layer is disposed by applying adhesive to the surface of the fiber lump portion 2. As the adhesive, various kinds of adhesives such as emulsion-based, thermosetting resin-based, hot melt-based, and elastomer-based adhesives can be used. It is particularly preferred to use emulsion-based or hot melt-based adhesive which is easy to handle. As the emulsion-based adhesive or hot melt-based adhesive, there are, for example, polyvinyl acetate, ethylene vinyl acetate copolymer, acrylic resin, and urethane resin. The adhesive application can be carried out by using, for example, spray method, curtain flow coating method, or dipping method.

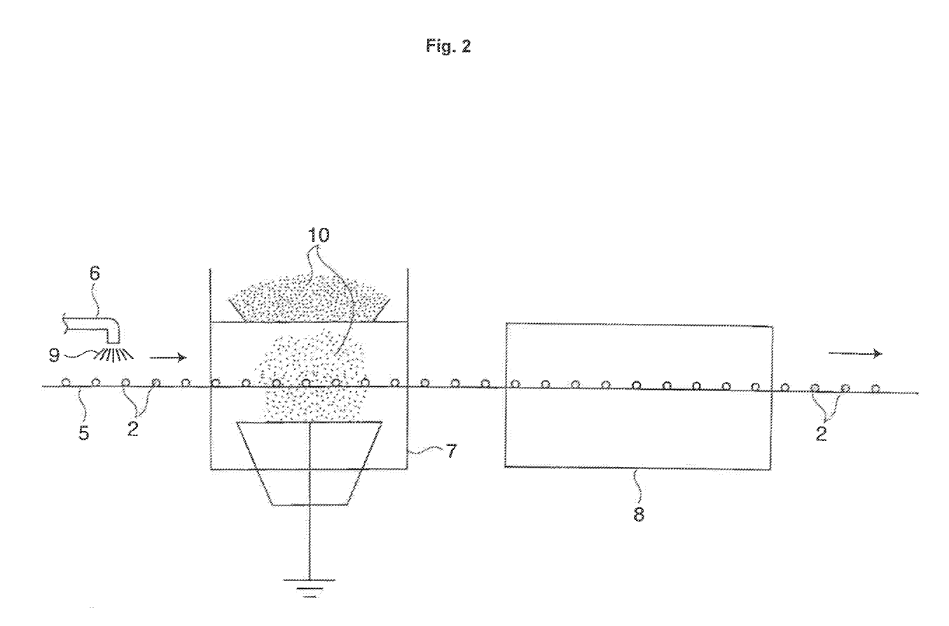

Next, the flocking method for disposing the flocked layer 3 by flocking process is described based on FIG. 2. FIG. 2 shows an example of the flocking step in the present preferred embodiment, in which the stick 4 having the fiber lump portion 2 disposed at one end or both ends thereof is conveyed by a belt conveyor 5 in one direction indicated by the arrow. An adhesive application nozzle 6, a flocking chamber 7, and a drying chamber 8 are installed in this order along the conveyance direction on a conveyance passage. The stick 4 is preferably conveyed while being rotated over the belt conveyor 5, with the axis thereof as the rotation center, in order to form the uniform flocked layer 3.

The adhesive application in the present embodiment is carried out by the application method called curtain flow coating method. That is, while allowing the adhesive 9 to flow from the nozzle 6, the fiber lump portion 2 is passed across the curtain flowing down therefrom so as to apply a certain amount of the adhesive to the surface of the fiber lump portion 2. The application amount can be adjusted by the passage speed of the fiber lump portion 2, the rotation speed of the stick 4, the viscosity of the adhesive, the opening area of the nozzle 6, or the like.

A pair of electrodes is disposed vertically across the belt conveyor 5 in the flocking chamber 7, and a DC high voltage of 20000 V to 40000 V is applied between the pair of electrodes. Then, the short fibers 10 are subjected to electrostatic force upon the insertion thereof between these two electrodes, and the short fibers fly in parallel with an electric field. The fiber lump portion 2 with the adhesive applied thereto is passed while being rotated into the space where the short fibers 10 fly, so that the short fibers 10 are substantially vertically attached to the surface of the fiber lump portion 2. Subsequently, the adhesive on the surface of the fiber lump portion 2 is dried and solidified or cured in the drying chamber 8, thereby forming the brush-shaped flocked layer 3.

When the hot melt-based adhesive is used as the adhesive, a cooling chamber or a blowing chamber is installed instead of the drying chamber 8, and the hot melt-based adhesive is cooled and solidified.

The swab of the present invention thus obtained has moderate soft elasticity owing to the fiber lump portion 2 disposed at one end of the stick 4. The brush-shaped flocked layer 3 formed on the surface of the fiber lump portion 2 has excellent fluid absorption capability and therefore quickly absorbs and retains the fluid, and also has superior fluid release performance.

Additionally, the surface texture is improved by the flocked layer 3.

Accordingly, the swab of the present invention is applicable to various kinds of uses conventionally employed. For example, the swab is suitable for taking biological specimens by inserting it into the mouth cavity, nasal cavity, eyes, urethra, or vagina of patients. The stimulation or pain exerted on the patients can be reduced by the elasticity of the fiber lump portion 2. The specimen absorbed and retained by the flocked layer 3 of the fiber lump portion 2 can be dissolved or dispersed into a culture fluid, for example, by immersing the fiber lump portion 2 into the culture fluid, followed by slight shaking. The specimen can also be released into a solid culture medium by moving the fiber lump portion 2 while bringing it into contact with the surface of the solid culture medium.

In the swab for taking the specimens according to the present invention, the fiber lump portion 2 is, of course, formed into the optimum shape according to the portion from which a specimen is taken. For example, the swab formed into a spatula shape is preferably used for mouth cavity.

The swab of the present invention is also suitable for applying a chemical agent to the affected part. Particularly, the swab has the moderate elasticity, and the flocked portion 3 on the surface thereof has a soft texture and feeling. Hence, the swab is less liable to give pain or irritation to the patient during the application. Further, owing to the excellent chemical agent absorbing performance and release performance of the flocked portion, the disadvantage that the chemical agent mostly permeates the fiber lump portion 2 and remains there during the application can be improved, thus permitting the effective use of the chemical agent.

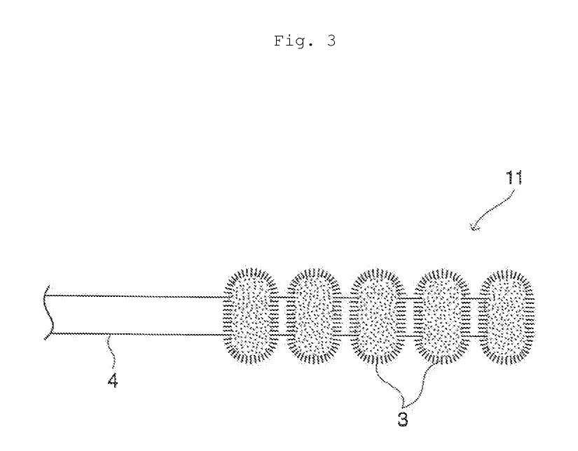

The swab of the present invention is also suitable for cleaning human bodies such as ears, as being usually used, because the flocked portion 3 has the soft texture and feeling. When the swab of the present invention is used as an ear cleaning stick, the swab provides an excellent effect of scraping together dirt by the brush-shaped flocked portion 3 on the surface thereof. For example, as shown in FIG. 3, when the flocked layer having the short fibers flocked thereon is disposed on the surfaces of ring-shaped fiber lump portions, the swab becomes suitable for use as, for example, an infant ear cleaning stick.

Further, the swab of the present invention is also suitable for cleaning equipment such as precision equipment and precision electronic equipment. That is, when the surface of the equipment is cleaned by impregnating cleaning fluid, such as solvent, into the fiber lump portion 2, the swab of the present invention provides an excellent effect of wiping dirt owing to the brush-shaped flocked portion 3 on the surface thereof.

Furthermore, the swab of the present invention is suitably usable as a cosmetic applicator for applying cosmetic materials, such as mascara, owing to the moderate elasticity and the soft texture, in addition to the excellent fluid retention performance and fluid release performance.

Next, the swab of the present invention is described in more detail with reference to an example. It is to be understood that the present invention is not limited to the following example.

### Example

According to the ordinary method, a cotton fiber lump portion having the shape as shown in FIG. 1 was formed at one end of a paper stick having a diameter of approximately 2 mm by using cotton fiber. This fiber lump portion had the maximum diameter portion of approximately 6 mm, and had the entire length of approximately 15 mm. According to the flocking step as shown in FIG. 2, ethylene vinyl acetate copolymer resin (EVA) based hot melt resin was applied to the surface of the fiber lump portion. Subsequently, in the flocking chamber, nylon short fibers (1.7 dtex×0.7 mm) were flocked in the brush-shape so as to cover the entire surface of the fiber lump portion. This was then conveyed to the blowing chamber, and the hot melt resin was cooled and solidified, thereby obtaining the flocked swab.

The elastic force in the flocked portion of the flocked swab obtained in the foregoing example was examined. As a result, it was confirmed that the elastic force thereof was substantially equal to the elastic force of the fiber lump portion before flocking. Consequently it is known that the flocked swab of the present example has not only a soft texture but also excellent elastic force.

While preferred embodiments of the present invention have been described and illustrated above, it is to be understood that the present invention is not limited to the foregoing preferred embodiments, and various improvements and changes can be made thereto within the scope of the following claims. For example, although the foregoing embodiment illustrates the swab in which the fiber lump portion 2 covered with the flocked layer 3 is disposed at one end of the stick 1, the fiber lump portion 2 covered with the flocked layer 3 may be similarly disposed at the other end of the stick 1. Alternatively, the flocked layer 3 is not necessarily required to cover the entire surface of the fiber lump portion 2. That is, only the necessary portion (for example, the front end) may be covered with the flocked layer 3 by partially masking the fiber lump portion 2 with a tape or the like.

### INDUSTRIAL APPLICABILITY

The swab of the present invention has a great potential for industrial applications thereof for taking biological specimens, or applying chemical agents to the affected parts, as well as for cleaning human bodies or equipment, or applying cosmetic materials.

What is claimed is:

1. A swab, comprising:
a stick;
a fiber lump portion disposed at one end or both ends of the stick; and
a flocked layer covering the surface of the fiber lump portion by flocking thereon fiber in a brush shape.

2. The swab according to claim 1, wherein the flocked layer partially or entirely covers the surface of fiber lump portion.

3. The swab according to claim 1, wherein the short fiber which forms the flocked layer is the same as or different from the fiber of the fiber lump portion, and comprises one kind, or a mixture of two or more kinds selected from the group consisting of rayon, polyester, polyamide, carbon fiber, cotton, and silk.

4. The swab according to claim 1, wherein the short fiber which forms the flocked layer is conductive fiber.

5. The swab according to claim 1, wherein the short fiber which forms the flocked layer has a fiber length of 0.3 to 3 mm, and a fiber thickness of 0.1 to 5.6 dtex.

6. The swab according to claim 1, wherein an adhesive layer is disposed on the surface of the fiber lump portion, and one end of the fiber for forming the flocked layer is adhered to the surface of the fiber lump portion by the adhesive layer.

7. The swab according to claim 1, intended for taking a biological specimen, applying a chemical agent to an affected part, cleaning a human body or equipment, or applying a cosmetic material.

8. The swab according to claim 1, wherein the fiber lump portion is a flat or curved plate shape, circular cylinder, circular cone, globular form, drop shape, or a circular cylinder with a plurality of circular grooves formed therein.
